(19) United States
(12) Patent Application Publication
(10) Pub. No.: US 2024/0252356 A1
(43) Pub. Date: Aug. 1, 2024

(54) MICROKERATOME BLADE ASSEMBLIES HAVING CHANNELS FOR STABILIZING MOVEMENT

(71) Applicant: Med-Logics, Inc., Athens, TX (US)

(72) Inventors: Rodney Ross, Larue, TX (US); Hoyt Ross, Athens, TX (US); Connor Bringman, Athens, TX (US); Clay McPherson, Tyler, TX (US)

(21) Appl. No.: 18/102,693

(22) Filed: Jan. 28, 2023

(51) Int. Cl. A61F 9/013

(52) U.S. Cl. CPC A61F 9/0133

(57) ABSTRACT

Microkeratome blade assemblies having channels for stabilizing movement are disclosed. According to an aspect, a microkeratome blade assembly including a blade having a cutting edge. The microkeratome blade assembly includes a blade holder attached to the blade. The blade holder defines one or more surfaces for moving across a support portion of a microkeratome head during operation of the blade. The blade holder defines a channel that extends substantially along the same direction as the cutting edge. The channel is shaped and sized for fitting to a corresponding protrusion formed in the microkeratome head.

### CROSS REFERENCE TO RELATED APPLICATION

This application is related to U.S. Design patent application No. ______, filed simultaneously herewith, and titled MICROKERATOME BLADE CARTRIDGE.

### TECHNICAL FIELD

The presently disclosed subject matter relates generally to medical devices for cutting a cornea. Particularly, the presently disclosed subject matter relates to microkeratome blade assemblies having channels for stabilizing movement.

### BACKGROUND

Laser in situ Keratomileusis (LASIK) is an eye surgical technique commonly used for correcting myopia and hyperopia, and for curing astigmatism. This technique is performed by an ophthalmologist who uses a laser or microkeratome to reshape the eye's cornea for improving visual acuity. LASIK can provide an alternative to the person wearing eyeglasses or contact lenses for correcting a vision problem.

Use of a microkeratome for a LASIK procedure involves placing a vacuum ring onto a cornea. A drive mechanism of the microkeratome is used to move a blade across the cornea while the cornea is held by the vacuum ring. The blade is moved in a reciprocating transverse direction to cut the eye. This creates a lamella flap of the cornea which is flipped back so that the stromal bed of the cornea can be ablated with a laser for correcting the vision problem.

Microkeratomes generally include three components: a hand piece that contains two motors, a head that holds the blade, and a vacuum ring that applied a suction to maintain the position of the microkeratome relative to the cornea. Precision of the procedure is very important to ensure that the cornea is cut properly. Therefore, stabilization of any of the components of the microkeratome during the operation is beneficial

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a perspective view of a microkeratome in accordance with the present disclosure;

FIG. 2 is a side view of the microkeratome shown in FIG. 1;

FIG. 3 is a front view of a lower portion of the microkeratome without the head so that a blade can be viewed;

FIG. 4 illustrates a top perspective view of the head, the blade, and the blade holder;

FIG. 5 is a side view of the blade assembly in operational position within the microkeratome head; and

FIGS. 6-10 are other views of the blade assembly and the microkeratome head.

### SUMMARY

The presently disclosed subject matter relates to microkeratome blade assemblies having channels for stabilizing movement. According to an aspect, a microkeratome blade assembly including a blade having a precise cutting edge. The microkeratome blade assembly includes a blade holder attached to the blade. The blade holder defines one or more surfaces for moving across a support portion of a microkeratome head during operation of the blade. The blade holder defines a channel that extends substantially along the same direction as the cutting edge. The channel is shaped and sized for fitting to a corresponding protrusion formed in the microkeratome head.

According to another aspect, a microkeratome includes a microkeratome head including a support portion. The microkeratome head also defines a protrusion. The microkeratome blade assembly also includes a blade including a cutting edge. Further, the microkeratome blade assembly includes a blade holder attached to the blade. The blade holder defines one or more surfaces for moving across the support portion of the microkeratome head during operation of the blade. The blade holder defines a channel that extends substantially along the same direction as the cutting edge. Further, the channel is shaped and sized for fitting to the protrusion formed in the microkeratome head.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

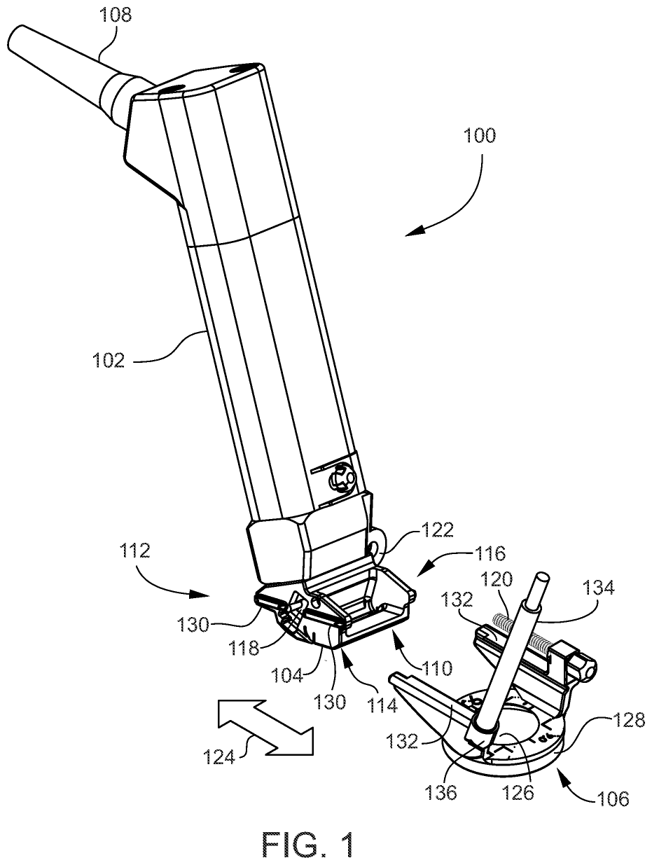

FIG. 1 illustrates a perspective view of a microkeratome 100 in accordance with the present disclosure. Referring to FIG. 1, the microkeratome 100 includes a hand piece 102 that is connected to a head 104 and a vacuum ring assembly 106. The microkeratome 100 may be used for cutting a lamella flap in a cornea as part of a LASIK procedure. The vacuum ring assembly 106 may be attached to a source of vacuum to create a suction pressure between the vacuum ring assembly 106 and the cornea. The suction pressure fixes the microkeratome 100 to the cornea. The hand piece 102 can include a wire assembly 108 that is connected to an electrical console, which is not shown for ease of illustration but should be understood by those of skill in the art. The console can provide electrical power to actuate the microkeratome 100.

The head 104 generally includes a front side 110, a rear side 112, a bottom side 114, and a top side 116. The head 104 defines a blade cavity 118 that can receive a blade, or a blade and a blade holder to which the blade is attached (not shown). The vacuum ring assembly 106 can include a helical gear 120 that can be coupled to the hand piece 102. For example, the helical gear 120 can be coupled to an internal gear (not shown) of the hand piece 102 at a coupling location 122. The hand piece 102 includes a motor (not shown) that cooperates with the helical gear 120 to move the head 104 and blade in a linear direction (indicated generally by double arrow 124) across an opening 126 of the vacuum ring assembly 106. The opening 126 is defined through a top surface 128 of the vacuum ring assembly 106 that faces the bottom side 114 of the head 104 and its blade. The cornea may protrude through the opening 126.

To accurately guide the head 104 and the blade along the linear direction 124, the head 104 and the vacuum ring assembly 106 can have one or more corresponding linear bearing members such as, for example, one or more corresponding tongues 130 and grooves 132, respectively, that create linear bearings. In this example, the tongues 130 are formed on the head 104, and the grooves 132 are formed on the vacuum ring assembly 106. In another example, the tongues 130 can be formed on the vacuum ring assembly 106, and the grooves 132 can be formed on the head 104. In another example, the head 104 can include a tongue 130 that movably engages a corresponding groove 132 of the vacuum ring assembly 106, and the head 104 can also include a groove 132 that is engaged by a corresponding tongue 130 of the vacuum ring assembly 106.

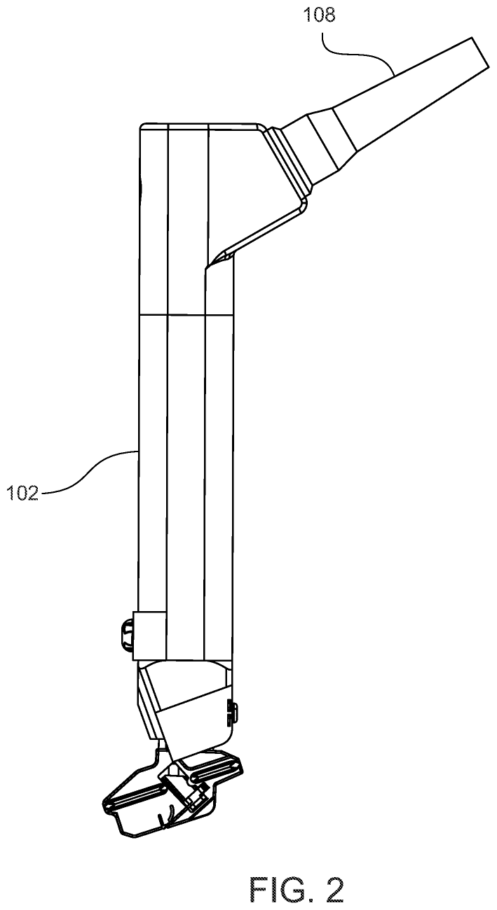

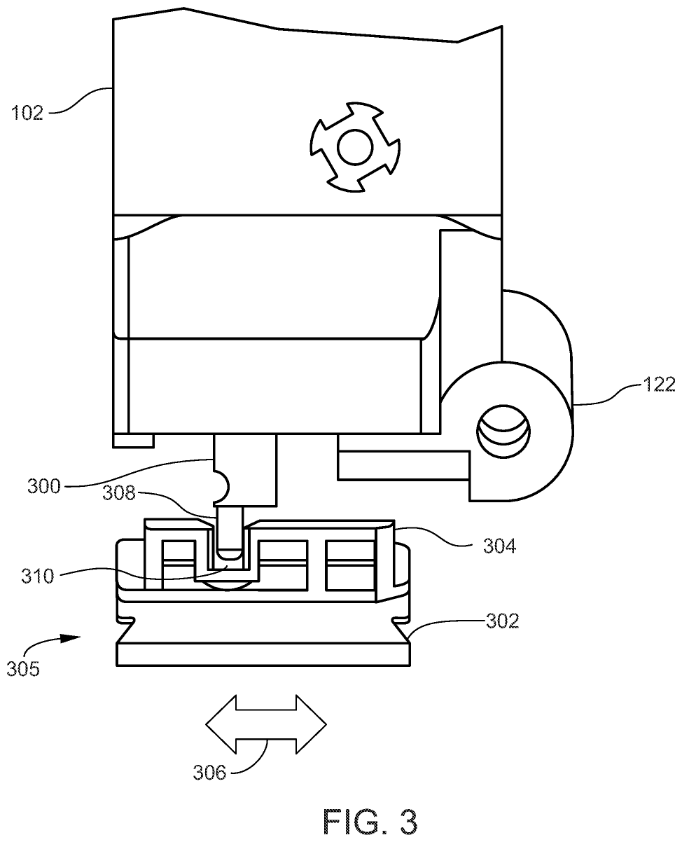

FIG. 2 illustrates a side view of the microkeratome 100 shown in FIG. 1. FIG. 2 shows a side of the microkeratome 100 that opposes the side shown in FIG. 1. In the side view of FIG. 2, it can be seen that the cavity 118 extends between the sides of the microkeratome 100. The hand piece 102 can include another motor (not shown due to its internal placement) that moves the blade in a lateral reciprocating (or oscillating) manner (i.e., orthogonal or transverse to the linear direction 124 along which the head 104 moves) so that the blade cuts corneal tissue and creates a lamella flap. For example, FIG. 3 illustrates a front view of a lower portion of the microkeratome 100 without the head 104 so that a blade 302 can be viewed. Referring to FIG. 3, this other motor may drive the rotation of a shaft 300. When the head 14 is attached to the hand piece 12 in this example, the shaft 300 extends through a coupling member (not shown) generally disposed at or near the top side 116 of the head 104 for engaging the blade 302 (or blade holder). The coupling member or some other portion of the structure of the head 104 may suitably attach the head 104 to, and detach the head 104 from, the hand piece 102. The blade 302 is attached to a blade holder 304 to form a blade assembly 305. The blade 302 may be made of metal, and the base 304 may be made of plastic or any other suitable medical grade material. The blade 302 and the blade holder 304 are sized and shaped for insertion into and positioning with the blade cavity 118 as described in more detail herein. Movement of the blade 302 along with the blade holder 304 during operation is indicated by double arrow 306.

Turning again to FIG. 1, the vacuum ring assembly 106 can include an aspiration connector 134. The aspiration connector 134 is connected to an aspiration tube (not shown) and is coupled to aspiration openings (not shown) in the vacuum ring. The aspiration connector 134 may have a collar 136 that limits the travel of the head 104 and the blade 302. The aspiration connector 134 may have a threaded shaft (not shown) that screws into a corresponding threaded opening (not shown) of the vacuum ring assembly 106.

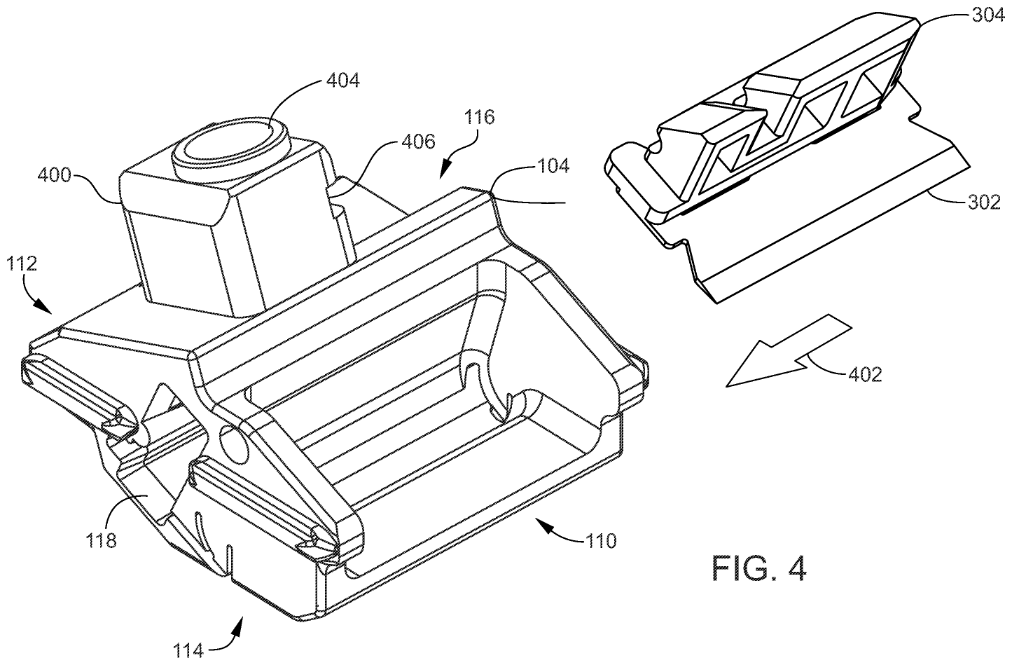

FIG. 4 illustrates a top perspective view of the head 104, the blade 302, and the blade holder 304. Referring to FIG. 4, the head 104 includes a coupling member 400 that can receive the shaft 300 shown in FIG. 3. In this figure, the blade 302 and the blade holder 304 are shown as being apart from the head 104 for ease of illustration. In order to be positioned for operation with the head, the blade 302 and the blade holder 304 may be moved generally moved in the direction indicated by arrow 402 until it is in the position as shown in FIG. 3. In this way, the blade 302 and the blade holder 304 may enter the blade cavity 118.

The shaft 300 can extend through the coupling member 400. Particularly, the coupling member 400 can define an opening 404 for insertion of the shaft 300. The coupling member 400 or some other portion of the structure of the head 104 can define a groove 406 utilized to attach the head 104 to, and detach the head 104 from, the hand piece 102.

The shaft 300 can engage the blade 302 for oscillating the blade 302 in the directions indicated by arrow 306 when the shaft 300 is rotated. Referring to FIG. 3, an eccentric pin 308 at an end of the shaft 300 can engage a slot 310 of the blade holder 304. Rotation of the shaft 300 causes the eccentric pin 308 to move the blade holder 304 and the blade 302 along the directions indicated by arrow 306.

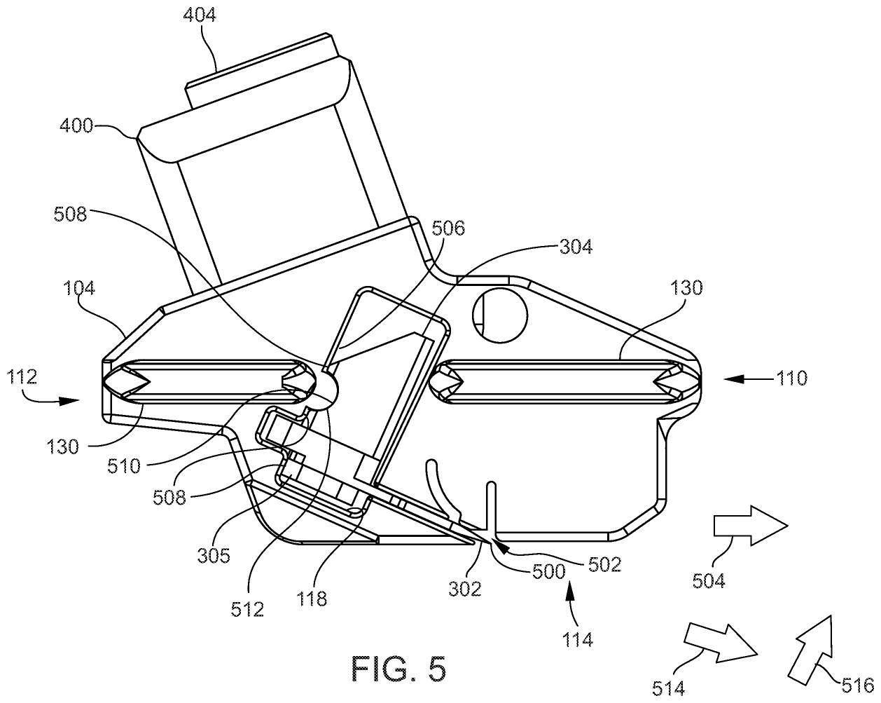

FIG. 5 illustrates a side view of the blade assembly 305 in operational position within the microkeratome head 104. Referring to FIG. 5, the blade 302 includes a cutting edge 500 that extends through an opening 502 at the bottom side 114 of the microkeratome head 104. The cutting edge 500 is positioned to engage and cut a cornea when the microkeratome head 104 is advanced forward in the direction indicated by arrow 504. The cutting edge 500 is oscillated in a direction orthogonal to this direction (indicated by arrow 504) when cutting the cornea.

When engaging the cornea, the blade assembly 305 is pushed rearward (in a direction generally opposing the direction of arrow 504). As a result, a rear portion of the blade assembly 305 engages a rear side 506 of the blade cavity 118. Particularly, surfaces 508 of the blade holder 304 contact the rear side 506 of the blade cavity 118 and can slide across the rear side 506 as the blade assembly 305 oscillates. The surfaces of the blade cavity 118 contacting the blade assembly 305 as shown function and may be referred to as part of a support portion of the microkeratome head 104. For example, one or more surfaces of the rear side of the blade cavity 118 can be support for the blade assembly 305 during a cutting operation.

The blade assembly 305 may be held in place during operation by the shaft 300. FIG. 3 shows that the shaft 300 in position for hold within the slot 310. The protruding offset cylindrical portion of the eccentric pin 308 mates with the slot 310 in the blade holder 304 to prevent it from being displaced unless the blade assembly 305 is removed from the hand piece 102 and shaft 300. Additionally, the shaft 300 may be spring loaded such that if it is not aligned with the slot 310 when the head is attached to the hand piece 102, it will seat itself once it starts spinning as the spring pushes it down when the eccentric pin 308 overlaps the slot 310. When these parts are properly mated, the blade 302 cannot move unless the shaft 300 is spinning.

Stability of the blade assembly 305 is beneficial to provide precise cutting of the cornea. For improved stability, the blade holder 304 defines a channel 510 that extends substantially along the same direction as the cutting edge 500. The channel 510 is shaped and sized for fitting to a corresponding protrusion 512 formed in the microkeratome head 104. The protrusion 512 fits tightly to the channel 510 so that stabilization of the blade assembly 305 is increased. Further, the protrusion 512 fits tightly to the channel 510 along its length such that stability is provided the entire distance of motion while the blade assembly 305 oscillates.

The channel 510 is substantially curved in shape. Also, the channel 510 is concave in shape. Although, the channel 510 may alternatively be any other suitable shape and size. For example, the channel 510 may be trapezoidal, square, or triangular in shape The channel 510 extends between sides of the blade holder 304.

It is noted that, in the alternative, the channel 510 and protrusion 512 may be exchanged. In other words, rather than a channel 510 being formed in the blade holder 304, it may be replaced with a protrusion similar to the protrusion 512. Further, in this example, the protrusion 512 may be replaced with a channel similar to the channel 510. Operation in this example may be similar to that described in other examples provided herein.

The protrusion 512 formed in the microkeratome head is convex in shape. In this example, the protrusion 512 is curved and shaped such that it fits closely to the shape of the channel 510. In other examples and so that it will fit to a correspondingly-shaped channel, the protrusion 512 may be trapezoidal, square, or triangular in shape.

With continuing reference to FIG. 5, the cutting edge 500 faces a direction generally in the direction of arrow 514. The channel 510 defines a smooth surface as shown, but it may be considered to have portions that faces in multiple different directions. At least one of the portions of the channel 510 faces a direction that substantially opposes the direction of arrow 514. Other directions that the surface portions face include a direction of arrow 516 or a direction opposing arrow 516 (orthogonal to the direction of arrow 514). Also, the surface portions face directions between the direction of arrow 516 and the direction opposing arrow 516. In this way, the channel 510 can securely fit to the corresponding shape of protrusion 512 for stabilizing blade movement. Also, it is noted that in this example the surface portions and smooth due to the curved shape, but alternatively, the surfaces may not be smoothly connected and have more distinct shape, such as a hexagonal-type shape.

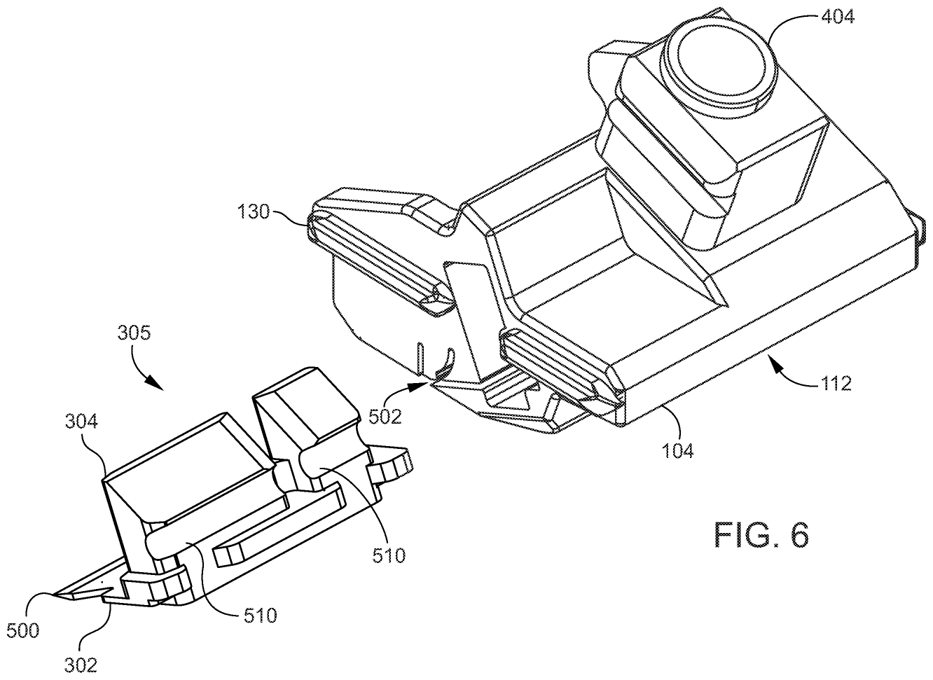

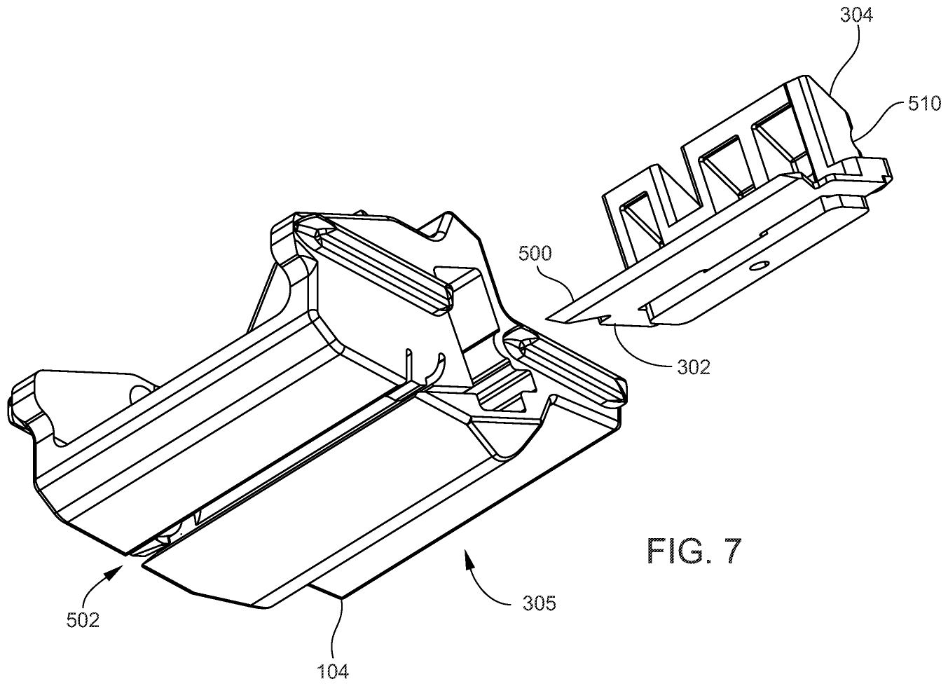

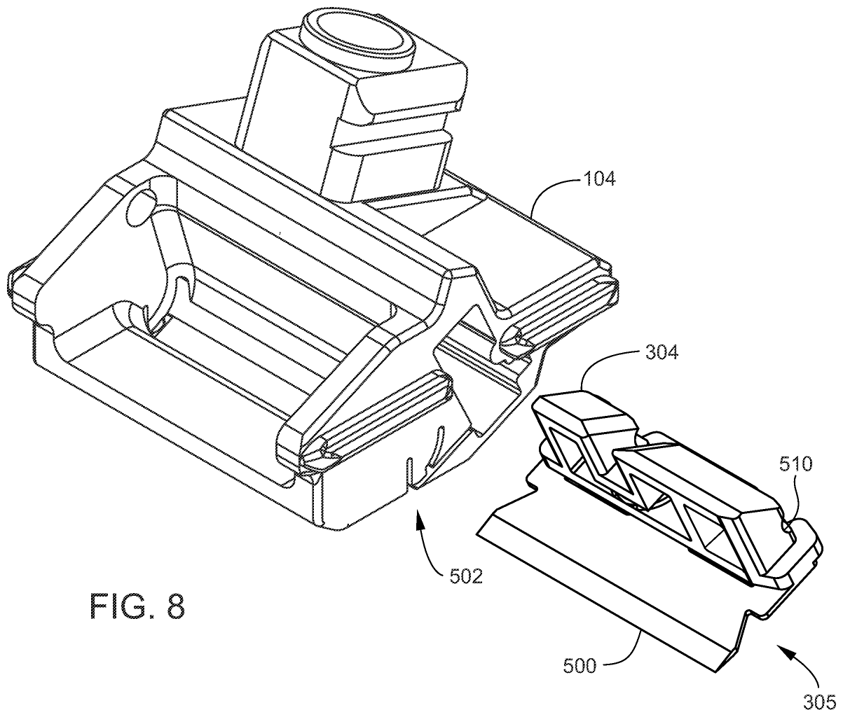

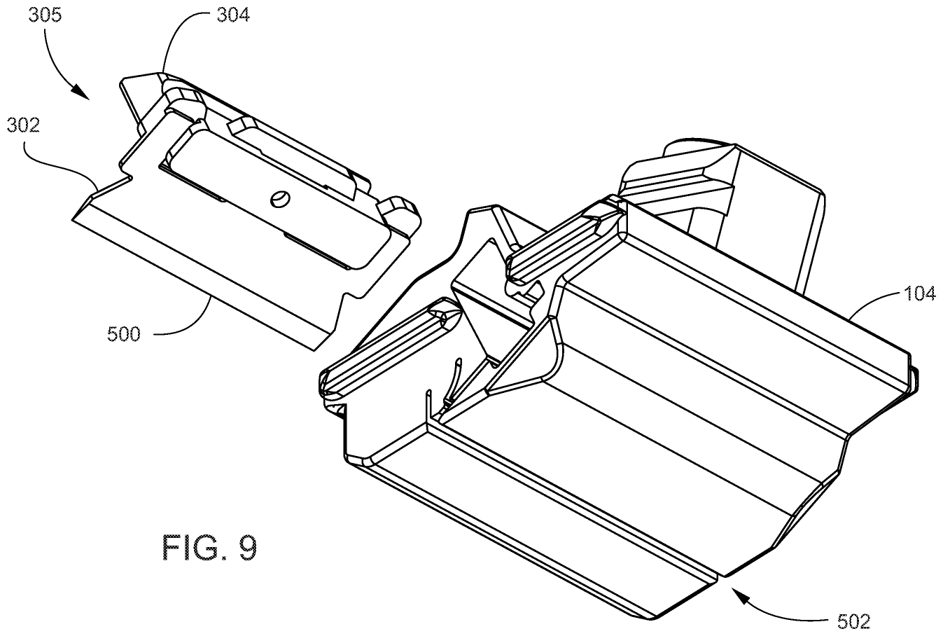

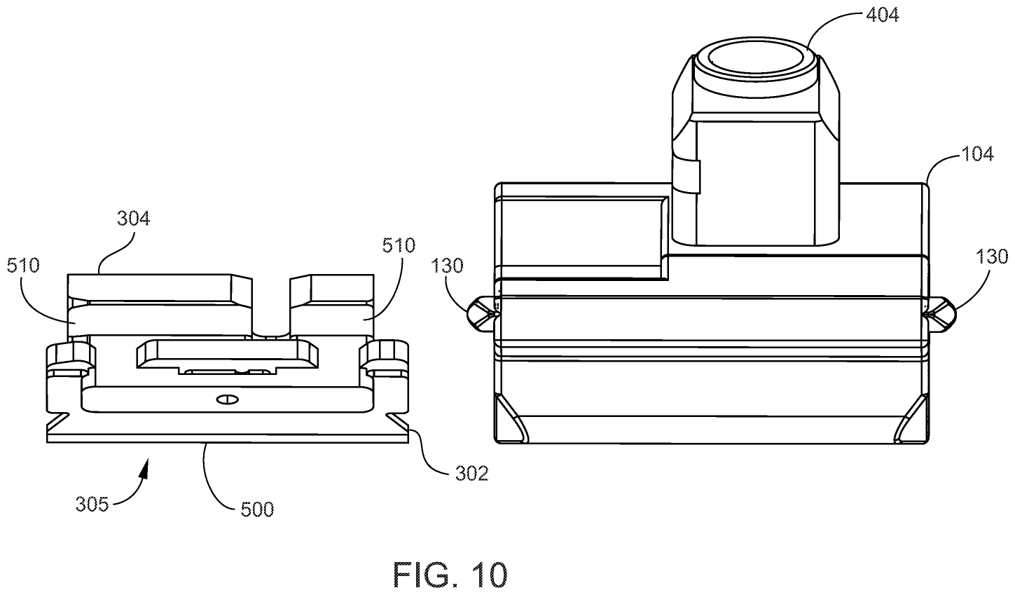

FIGS. 6-10 illustrate other views of the blade assembly 305 and the microkeratome head 104. For ease of illustration, the blade assembly 305 is positioned apart from the microkeratome head 104. FIG. 6 illustrates a rear, top perspective view, FIG. 7 illustrates a bottom perspective view, FIG. 8 illustrates a front, top perspective view, FIG. 9 illustrates another bottom perspective view, and FIG. 10 illustrates a rear view.

It is noted that the microkeratome may be of any suitable material. For example, the head may be made of a stainless steel material that is electroplated with aluminum (e.g., MEDCOAT 2000™ by The Electrolizing Corporation of Ohio). The blade may be made of stainless steel and conforming to the ASTM A-176 standard. The blade holder may be made of copolymer or polyoxymethylene copolymer.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A microkeratome blade assembly comprising:
a blade including a cutting edge; and
a blade holder attached to the blade, wherein the blade holder defines one or more surfaces for moving across a support portion of a microkeratome head during operation of the blade, wherein the blade holder defines a channel that extends substantially along the same direction as the cutting edge, and wherein the channel is shaped and sized for fitting to a corresponding protrusion formed in the microkeratome head.

2. The microkeratome blade assembly of claim 1, wherein the cutting edge is substantially straight.

3. The microkeratome blade assembly of claim 1, wherein the channel extends in a direction substantially parallel to the direction of operational motion of the blade.

4. The microkeratome blade assembly of claim 1, wherein the channel is curved, trapezoidal, square, or triangular in shape.

5. The microkeratome blade assembly of claim 1, wherein the channel is concave in shape.

6. The microkeratome blade assembly of claim 1, wherein the corresponding protrusion formed in the microkeratome head is curved, trapezoidal, square, or triangular in shape.

7. The microkeratome blade assembly of claim 1, wherein the corresponding protrusion formed in the microkeratome head is convex in shape.

8. The microkeratome blade assembly of claim 1, wherein the cutting edge faces a first direction, and
wherein the channel defines a surface having portions that face a plurality of directions, and wherein one of the plurality of directions is a second direction that substantially opposes the first direction.

9. The microkeratome blade assembly of claim 8, wherein at least one of the plurality of directions is a third direction that is orthogonal to the first direction.

10. The microkeratome blade assembly of claim 8, wherein the corresponding protrusion has a plurality of surfaces that face in directions that substantially oppose directions faced by the surface of the channel.

11. A microkeratome comprising:
a microkeratome head including a support portion and that defines a protrusion; and
a microkeratome blade assembly comprising:
a blade including a cutting edge; and
a blade holder attached to the blade, wherein the blade holder defines one or more surfaces for moving across the support portion of the microkeratome head during operation of the blade, wherein the blade holder defines a channel that extends substantially along the same direction as the cutting edge, and wherein the channel is shaped and sized for fitting to the protrusion formed in the microkeratome head.

12. The microkeratome of claim 11, wherein the cutting edge is substantially straight.

13. The microkeratome of claim 11, wherein the channel extends in a direction substantially parallel to the direction of operational motion of the blade.

14. The microkeratome of claim 11, wherein the channel is curved, trapezoidal, square, or triangular in shape.

15. The microkeratome of claim 11, wherein the channel is concave in shape.

16. The microkeratome of claim 11, wherein the corresponding protrusion formed in the microkeratome head is curved, trapezoidal, square, or triangular in shape.

17. The microkeratome of claim 11, wherein the corresponding protrusion formed in the microkeratome head is convex in shape.

18. The microkeratome of claim 11, wherein the cutting edge faces a first direction, and
wherein the channel defines a surface having portions that face a plurality of directions, and wherein one of the plurality of directions is a second direction that substantially opposes the first direction.

19. The microkeratome of claim 18, wherein at least one of the plurality of directions is a third direction that is orthogonal to the first direction.

20. The microkeratome of claim 18, wherein the corresponding protrusion has a plurality of surfaces that face in directions that substantially oppose directions faced by the surface of the channel.

21. A microkeratome comprising:
a microkeratome head including a support portion and that defines a channel; and
a microkeratome blade assembly comprising:
a blade including a cutting edge that extends substantially along the same direction as the channel; and
a blade holder attached to the blade, wherein the blade holder defines one or more surfaces for moving across the support portion of the microkeratome head during operation of the blade, wherein the blade holder defines a protrusion that is shaped and sized for fitting to the channel formed in the microkeratome head.

22. The microkeratome of claim 21, wherein the channel is curved, trapezoidal, square, or triangular in shape.
